(19) United States
(12) Patent Application Publication
(10) Pub. No.: US 2021/0212810 A1
(43) Pub. Date: Jul. 15, 2021

(54) MULTI-LAYER SKIN CONSTRUCTS AND METHODS OF MAKING AND USING THE SAME

(71) Applicant: Wake Forest University Health Sciences, Winston-Salem, NC (US)

(72) Inventors: Mathew VARKEY, Winston-Salem, NC (US); Anthony ATALA, Winston-Salem, NC (US)

(21) Appl. No.: 16/768,963

(22) PCT Filed: Dec. 7, 2018
(86) PCT No.: PCT/US2018/064471

§ 371 Date: Jun. 2, 2020

Related U.S. Application Data
(60) Provisional application No. 62/595,818, filed on Dec. 7, 2017.

(51) Int. Cl. A61F 2/10; A61L 27/18; A61L 27/38; A61L 27/60

(52) U.S. Cl. CPC A61F 2/105; A61L 27/18; A61L 2430/34; A61L 27/3891; A61L 27/60; A61L 27/3804

(57) ABSTRACT

Provided are skin constructs and methods of making and using the same, such as for wound treatment and/or compound testing, including compound testing for efficacy, toxicity, immune response, penetration, irritation and/or metabolism testing of drug candidates or compositions such as cosmetics.

### FIELD

The present invention concerns live, artificial, skin constructs and methods of making and using the same, such as for wound treatment and compound testing.

### BACKGROUND

The current “gold standard” for skin replacement is the use of autologous skin grafts. However, due to donor-site tissue availability, complex maintenance and costs of such tissues, this treatment is often limited for patients. Also, most current engineered skins or skin substitutes do not fully recapitulate native skin as they are devoid of multiple skin cell types and structures like trilayers and dermal appendages. The current commercially available skin cellular models are also limited as they only use either immortalized cell lines derived from skin tumors or one or two primary cell types (e.g., keratinocytes and/or dermal fibroblasts) to be simple; thus they do not well represent and replicate the complexity of in vivo skin.

E. Bellas et al., In vitro 3D full thickness skin equivalent tissue model using silk and collagen biomaterials, Macromol. Biosci 12, 1627-1636 (2012), utilize adipose derived stem cells, keratinocytes, and fibroblasts to create a tri-layer skin-like product, but require the use of a silk scaffold onto which cells are seeded.

A. Skardal et al., Bioprinted Amniotic Fluid-Derived Stem Cells Accelerate Healing of Large Skin Wounds, Stem Cells Translational Medicine 1, 792-802 (2012), describes bioprinting of a skin-substitute directly onto a large wound, but use only amniotic fluid stem cells and bone-marrow-derived mesenchymal stem cells.

A. Monfort et al., Production of a human tissue-engineered skin trilayer on a plasma-based hypodermis, J. Tissue Eng. Regen. Med. 7, 479-490 (2013), describes a skin-like trilayer product, but employed only adipogenic cells, fibroblasts, and keratinocytes, and used sequential culturing techniques that required 35 days to complete. Id. at 480-81.

V. Lee et al., Design and Fabrication of Human Skin by Three-Dimensional Bioprinting, Tissue Engineering 20, 473-484 (2014), describe a skin-like product, created with 3D bioprinting, but utilized only keratinocytes and fibroblasts, printed between separate collagen layers. See, e.g., FIG. 2 therein.

Yoo, Xu and Atala et al., US Patent Application Publication No. US 2009/0208466 (August 2009) suggests skin substitute products at page 3, paragraphs 0037-0041, but does not suggest or describe, for example, how papilla cells may be effectively incorporated therein.

PCT Publication WO 2016/115034 to Atala et al. (July 2016) describes skin substitute products with three layers and multiple cell types, including papilla cells. However, there remains a need for further improvements of skin substitute products to that can be used for therapeutic and/or drug testing purposes.

### SUMMARY

Provided herein is an artificial mammalian skin construct, comprising:

optionally, a first (“hypodermis-like”) layer comprising live mammalian adipocytes (e.g., induced pre-adipocytes) and optionally live mammalian endothelial cells (e.g., dermal microvasculature endothelial cells) in a first hydrogel carrier;

a second (“dermis-like”) layer on or directly contacting said first layer, when present, said second layer comprising live mammalian fibroblast cells, live mammalian follicle dermal papilla cells, and optionally live mammalian endothelial cells (e.g., dermal microvasculature endothelial cells) in combination in a second hydrogel carrier; and

a third (“epidermis-like”) layer on or directly contacting said second layer, said third layer comprising live mammalian keratinocytes, live mammalian melanocytes, and live mammalian immune cells (e.g., CD14+ monocytes, Langerhans cells, dermal dendritic cells, or a combination of two of more thereof) in combination in a third hydrogel carrier.

In some embodiments, the construct has visible pigmentation (e.g., after 3, 4, 5, 6, 7, or 8 weeks in culture).

In some embodiments, the live mammalian immune cells of the third layer comprise Langerhans cells and dermal dendritic cells (e.g., after 5 days in culture, and up to 3, 4, 5, 6, 7, or 8 weeks in culture).

In some embodiments, the hypodermis-like layer, the dermis-like layer, or both, comprise the live mammalian endothelial cells.

In some embodiments, both the hypodermis-like layer and the dermis-like layer comprise the live mammalian endothelial cells.

In some embodiments, the construct is a stratified, tri-layered construct.

In some embodiments, the construct has hair follicle structure organization (inner and outer root sheaths, which may be indicated by being cytokeratin 14 positive and cytokeratin 71 positive) in vitro, and/or are positive for PROMININ-1 (e.g., after 5 days in culture, and up to 3, 4, 5, 6, 7, or 8 weeks in culture).

In some embodiments, the construct is produced by a process comprising:

(a) optionally co-culturing the adipocytes and endothelial cells as spheroids; incorporating the spheroids into the first hydrogel carrier to form a hypodermal bioink, and depositing (e.g., by bioprinting) the hypodermal bioink on a substrate to form the first (hypodermis-like) layer;

(b) culturing the follicle dermal papilla cells as spheroids; independently, co-culturing the endothelial cells and the fibroblasts as spheroids; incorporating the spheroids into the second hydrogel carrier to form a dermal bioink, and depositing (e.g., by bioprinting) the dermal bioink onto the hypodermis-like layer, when present, or a substrate when not present, to form the second (dermis-like) layer; and

(c) incorporating the keratinocytes, the melanocytes and the immune cells into the third hydrogel carrier to form an epidermal bioink, and depositing (e.g., by bioprinting) the epidermal bioink onto the dermis-like layer to form the third (epidermis-like) layer,

to thereby form the skin construct.

In some embodiments, the construct is produced by a process comprising:

(a) incorporating the keratinocytes, the melanocytes and the immune cells into a third hydrogel carrier to form an epidermal bioink; and depositing (e.g., by bioprinting) the epidermal bioink on a substrate to form the third (epidermis-like) layer;

(b) culturing the follicle dermal papilla cells as spheroids; independently, co-culturing the endothelial cells and the fibroblasts as spheroids; incorporating the spheroids into the second hydrogel carrier to form a dermal bioink; and depositing (e.g., by bioprinting) the dermal bioink onto the epidermis-like layer to form the second (dermis-like) layer; and

(c) optionally, co-culturing the adipocytes and the endothelial cells as spheroids; incorporating the spheroids into the first hydrogel carrier to form a hypodermal bioink; and depositing (e.g., by bioprinting) the hypodermal bioink onto the dermis-like layer to form the first (hypodermis-like) layer,

to thereby form the skin construct.

In some embodiments, the depositing is carried out by bioprinting (e.g., “ink jet” type printing and/or syringe injection type printing).

Also provided is a method of treating a wound on a subject in need thereof, comprising topically applying a skin construct as taught herein to said wound in a treatment-effective amount and/or configuration. In some embodiments, the cells of the construct are autologous or allogeneic with respect to the subject.

In some embodiments, the skin construct further comprises an inert mold layer on or contacting said third layer. In some embodiments, the inert mold layer is dimensioned for custom fit onto a facial wound (e.g., based on scan data).

Also provided is a method of screening a compound or composition for activity when applied to the skin of a mammalian subject, comprising: providing a skin construct as taught herein under conditions which maintain constituent cells of said construct alive; contacting said compound or composition to said construct; and then detecting a response of said skin construct, the presence of such response indicating said compound or composition is potentially active if applied to the skin of a mammalian subject.

Further provided is a method of making a skin construct, comprising the steps of:

(a) optionally co-culturing adipocytes and endothelial cells as spheroids; incorporating the spheroids into a first hydrogel carrier to form a hypodermal bioink; and depositing (e.g., by bioprinting) the hypodermal bioink on a substrate to form a first (hypodermis-like) layer;

(b) culturing follicle dermal papilla cells as spheroids; independently, co-culturing endothelial cells and fibroblasts as spheroids; incorporating the spheroids into a second hydrogel carrier to form a dermal bioink; and depositing (e.g., by bioprinting) the dermal bioink onto the hypodermis-like layer, when present, or a substrate when not present, to form a second (dermis-like) layer; and

(c) incorporating the keratinocytes, melanocytes and immune cells into a third hydrogel carrier to form an epidermal bioink; and depositing (e.g., by bioprinting) the epidermal bioink onto the dermis-like layer to form a third (epidermis-like) layer,

to thereby make the skin construct.

Also provided is a method of making a skin construct, comprising the steps of:

(a) incorporating the keratinocytes, melanocytes and immune cells into a third hydrogel carrier to form an epidermal bioink; and depositing (e.g., by bioprinting) the epidermal bioink on a substrate to form a third (epidermis-like) layer;

(b) culturing follicle dermal papilla cells as spheroids; independently, co-culturing endothelial cells and fibroblasts as spheroids; incorporating the spheroids into a second hydrogel carrier to form a dermal bioink; and depositing (e.g., by bioprinting) the dermal bioink onto the epidermis-like layer to form a second (dermis-like) layer; and

(c) optionally, co-culturing adipocytes and endothelial cells as spheroids; incorporating the spheroids into a first hydro gel carrier to form a hypodermal bioink; and depositing (e.g., by bioprinting) the hypodermal bioink onto the dermis-like layer to form a first (hypodermis-like) layer,

to thereby make the skin construct.

In some embodiments, the depositing is carried out by bioprinting (e.g., “ink jet” type printing and/or syringe injection type printing).

In some embodiments, the substrate is an inert substrate.

In some embodiments, the substrate is a wound on a subject (e.g., a human subject) in need of treatment, and optionally wherein the cells are autologous or allogenic. Further provided is the use of a skin construct as taught herein in a method of treating a wound on a subject (e.g., a human subject) in need of treatment, and optionally wherein the cells are autologous or allogenic.

In some embodiments, the method further comprises culturing the skin construct in vitro under submerged conditions; then culturing at an air-liquid interface, with the epidermal-like layer exposed to air, for a time sufficient to facilitate epidermal stratification of the skin construct.

The present invention is explained in greater detail in the drawings herein and the specification set forth below.

### BRIEF DESCRIPTION OF THE DRAWINGS

FIG. 1 provides a schematic of forming skin constructs by bioprinting.

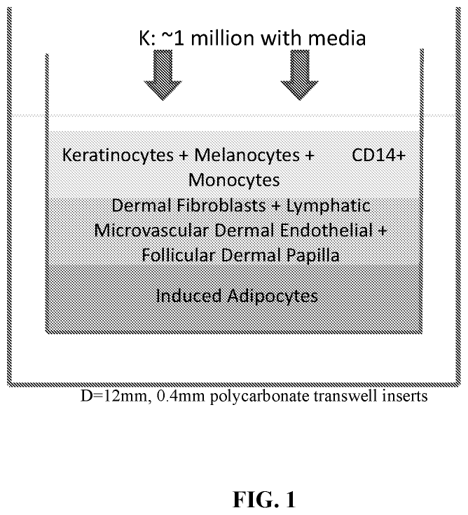

FIG. 2 is a photograph of skin constructs made in accordance with the present disclosure and having visible pigmentation.

FIG. 3 shows the layered configuration of bioprinted skin constructs at Day 4 and at Day 15.

### DETAILED DESCRIPTION

The present invention is explained in greater detail in the drawings herein and the specification set forth below. The disclosures of all United States patent references cited herein are incorporated by reference herein in their entireties.

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used herein is for the purpose of describing particular embodiments only and is not intended to be limiting of the invention. As used herein, the singular forms “a,” “an” and “the” are intended to include plural forms as well, unless the context clearly indicates otherwise. It will be further understood that the terms “comprises” or “comprising,” when used in this specification, specify the presence of stated features, integers, steps, operations, elements components and/or groups or combinations thereof, but do not preclude the presence or addition of one or more other features, integers, steps, operations, elements, components and/or groups or combinations thereof.

As used herein, the term “and/or” includes any and all possible combinations or one or more of the associated listed items, as well as the lack of combinations when interpreted in the alternative (“or”).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and claims and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

It will be understood that, although the terms first, second, etc., may be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section.

“Mammalian” as used herein refers to both human subjects (and cells sources) and non-human subjects (and cell sources or types), such as dog, cat, mouse, monkey, etc. (e.g., for veterinary or research purposes).

“Hydrogel” as used herein may be any suitable hydrogel. In general, the hydrogel includes water and is further comprised of or derived from polyalkylene oxides, poloxamines, celluloses, hydroxyalkylated celluloses, polypeptides, polysaccharides, carbohydrates, proteins, copolymers thereof, or combinations thereof, and more particularly are comprised of or derived from poly(ethylene glycol), poly(ethylene oxide), poly(vinyl alcohol); poly(vinylpyrrolidone), poly(ethyloxazoline), poly(ethylene oxide)-co-polypropylene oxide) block copolymers, carboxymethyl cellulose, hydroxyethyl cellulose, methylhydroxypropyl cellulose, polysucrose, hyaluronic acid, dextran, heparan sulfate, chondroitin sulfate, heparin, alginate, gelatin, collagen, albumin, ovalbumin, copolymers thereof, and combinations thereof, all of which are preferably cross-linked to varying degrees in accordance with known techniques, or variations thereof that are apparent to those skilled in the art. See, e.g., U.S. Pat. Nos. 8,815,277; 8,808,730; 8,754,564; 8,691,279. In some embodiments, a cross-linked hyaluronic acid hydrogel (optionally including additional polymers such as gelatin and/or collagen) is preferred.

### 1. Skin Constructs and Methods of Making the Same.

In some embodiments, skin constructs of the invention may be made by the steps of:

(a) optionally depositing a first (“hypodermis-like”) layer comprising live mammalian adipocytes (e.g., induced pre-adipocytes) and optionally live mammalian endothelial cells in a first hydrogel carrier on a substrate (e.g., an inert substrate such as a porous polymer mesh; collagen, etc.; or a wound on a subject in need of treatment);

(b) depositing a second (“dermis-like”) layer on said first layer when present (or on said substrate when said first layer is not present), said second layer comprising live mammalian fibroblast cells and live mammalian follicle dermal papilla cells, and optionally live mammalian endothelial cells, in a second hydrogel carrier; and

(c) depositing a third (“epidermis-like”) layer on said second layer, said third layer comprising live mammalian keratinocytes, live mammalian melanocytes and live mammalian immune cells (e.g., CD14+ monocytes) in a third hydrogel carrier.

The first, second and/or third hydrogel carriers may be the same, or may be different.

These steps may be reversed, i.e., depositing the epidermis-like layer, then the dermis-like layer, and optionally the hypodermis-like layer.

“Immune cells” as used herein includes, but is not limited to, CD14+ monocytes, Langerhans cells, dermal dendritic cells, or a combination of two of more thereof. In some embodiments, the immune cells include both Langerhans cells and dermal dendritic cells in the formed construct, e.g., after culture thereof for 3, 4, 5, 6, 7, or 8 or more weeks in vitro, in which the CD14+ monocytes may differentiate into both Langerhans cells and dermal dendritic cells.

In some embodiments, the hypodermis-like layer, the dermis-like layer, or both, include the live mammalian endothelial cells.

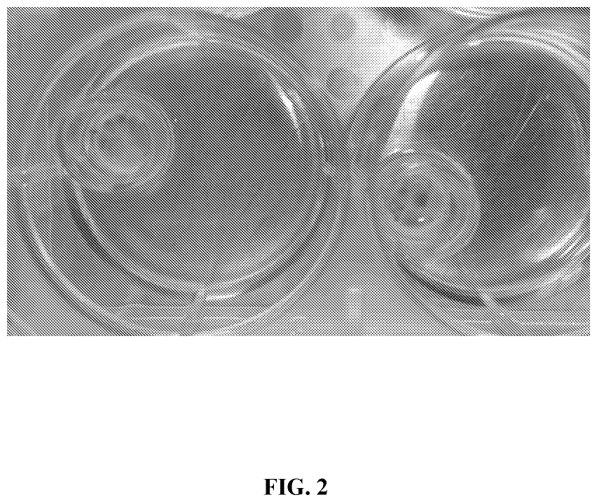

In some embodiments, the construct has visible pigmentation (e.g., after 3, 4, 5, 6, 7, or 8 weeks in culture), i.e., visible to the naked/unaided human eye (see FIG. 2).

In some embodiments, the construct is a stratified, tri-layered construct.

In some embodiments, the construct has hair follicle structure organization (inner and outer root sheaths, which may be indicated by being cytokeratin 14 positive and cytokeratin 71 positive, respectively) in vitro, and/or are positive for PROMININ-1 (indicating melanocytes/pigmentation).

In some embodiments, one or more cell types to be incorporated into the construct are provided and/or cultured as spheroids. In some embodiments, adipocytes are provided and/or cultured as spheroids. In some embodiments, endothelial cells are provided and/or cultured as spheroids. In some embodiments, adipocytes are provided and/or cultured as spheroids in co-culture with endothelial cells. In some embodiments, follicle dermal papilla cells are provided and/or cultured as spheroids. In some embodiments, fibroblasts are provided and/or cultured as spheroids. In some embodiments, fibroblasts are provided and/or cultured as spheroids in co-culture with endothelial cells. In some embodiments, keratinocytes are provided and/or cultured as spheroids. In some embodiments, melanocytes are provided and/or cultured as spheroids. In some embodiments, immune cells are provided and/or cultured as spheroids. In some embodiments, one or more cell types are not provided and/or cultured as spheroids. For example, in some embodiments, keratinocytes, melanocytes, and/or immune cells are not provided and/or cultured as spheroids.

“Spheroid” as used herein refers to a composition of live cells, typically in a carrier media, arranged in a three-dimensional or multi-layered configuration (as opposed to a two-dimensional or monolayer culture). Spheroid culturing may be performed, e.g., with appropriate cell cultureware. See, e.g., US 2014/0322806 to Bennett et al.

In some embodiments, a spheroid is about 100 μm, 200 μm, or 350 μm to about 500 μm, 750 μm or 1,000 μm in diameter, such as, for example, about 100, 150, 200, 250, 300, 350, 400, 450, 500, 550, 600, 650, 700, 750, 800, 850, 900, 950, or 1000 μm. The spheroid may comprise about 1,500, 2,000, 5,000, 10,000, 25,000, or 50,000 cells in total, to about 100,000, 500,000, 1 million, 2 million, or 5 million cells in total.

Suitable carrier media of the spheroids include compositions of the present invention (e.g., hydrogels, such as cross-linked hydrogels, of the present invention).

More particularly, in some embodiments the skin construct may be made by the steps of:

(a) optionally co-culturing adipocytes and endothelial cells as spheroids; incorporating the spheroids into a first hydrogel carrier to form a hypodermal bioink; and depositing (e.g., by bioprinting) the hypodermal bioink on a substrate to form the first (hypodermis-like) layer;

(b) culturing follicle dermal papilla cells as spheroids; independently, co-culturing endothelial cells and fibroblasts as spheroids; incorporating the spheroids into a second hydrogel carrier to form a dermal bioink; and depositing (e.g., by bioprinting) the dermal bioink onto the hypodermis-like layer, when present, or a substrate when not present, to form the second (dermis-like) layer; and

(c) incorporating the keratinocytes, melanocytes and immune cells into a third hydrogel carrier to form an epidermal bioink; and depositing (e.g., by bioprinting) the epidermal bioink onto the dermis-like layer to form the third (epidermis-like) layer.

Alternatively, in some embodiments the skin construct may be made by the steps of:

(a) incorporating the keratinocytes, melanocytes and immune cells into a third hydrogel carrier to form an epidermal bioink; and depositing (e.g., by bioprinting) the epidermal bioink on a substrate to form the third (epidermis-like) layer;

(b) culturing follicle dermal papilla cells as spheroids; independently, co-culturing endothelial cells and fibroblasts as spheroids; incorporating the spheroids into a second hydrogel carrier to form a dermal bioink; and depositing (e.g., by bioprinting) the dermal bioink onto the epidermis-like layer to form the second (dermis-like) layer; and

(c) optionally, co-culturing adipocytes and endothelial cells as spheroids; incorporating the spheroids into a first hydrogel carrier to form a hypodermal bioink; and depositing (e.g., by bioprinting) the hypodermal bioink onto the dermis-like layer to form the first (hypodermis-like) layer.

In some embodiments, the first hydrogel carrier, when deposited, is deposited in prepolymerized or partially polymerized form; the second hydrogel carrier is deposited in prepolymerized or partially polymerized form; and/or the third hydrogel carrier is deposited in prepolymerized or partially polymerized form.

In some embodiments, the depositing steps (a) and (b) are carried out under conditions in which said first hydrogel in said first layer, when present, and said second hydrogel in said second layer at least partially crosslink with one another; and/or said depositing steps (b) and (c) are carried out under conditions in which said second hydrogel in said second layer and said third hydrogel in said third layer at least partially crosslink with one another. The layers may be crosslinked directly, or through an intervening cross-linkable layer.

In some embodiments, the first, second, and/or third hydrogel carriers comprise cross-linked hyaluronic acid, and/or the second and/or third hydrogel carriers optionally but preferably further comprise gelatin and/or collagen.

In some embodiments, the depositing is carried out under conditions in which the second and third layers are at least partially cross-linked with one another, and/or the first layer and second layers are at least partially cross-linked with one another—typically by carrying out the depositing steps sufficiently close in time so that cross-linking reaction between the two layers may occur.

In some embodiments, partial or complete intervening layer(s), e.g., intervening hydrogel layer(s), can be interposed between the first and second hydrogen layers, and/or the second and third hydrogel layers, with the first and second, and/or second and third, hydrogel layers optionally cross-linked with their respective intervening hydrogel layer(s). By “partial” intervening layer is meant that the layer has openings therein through which the first and second, and/or second and third, layers directly contact one another. In addition, additional cell types such as described below may optionally be deposited with such intervening layers. The hydrogels of these intervening layer(s), when present, may be formed of the same materials as the first, second, and/or third hydrogel layers, and like those layers may be deposited in partially crosslinked form.

In some embodiments: (i) said first layer, when present, has a thickness of from 100, 200 or 300 micrometers up to 400, 600 or 800 micrometers; (ii) said second layer has a thickness of from 100, 200 or 300 micrometers up to 400, 600 or 800 micrometers; (iii) said third layer has a thickness of from 100, 200 or 300 micrometers up to 400, 600 or 800 micrometers; and/or (iv) said construct has a total thickness of from about 200, 400 or 600 micrometers up to 800, 1200 or 1600 micrometers when said first layer is absent, or a total thickness of 300, 600 or 900 micrometers up to 1200, 1800 or 2400 micrometers when said first layer is present.

In some embodiments, each of the first layer when present, said second layer, and said third layer have overlying surface areas of from 0.5, 1 or 10 square centimeters up to 50, 200 or 400 square centimeters.

Cells may be included in any suitable amount. In some embodiments: (i) said adipocytes (and endothelial cells, if present) are included in said first hydrogel carrier in an amount of from 1 or 2 million to 8, 10, 15 or 20 million (preferably 4 to 6 million or 10 to 20 million) cells per cubic centimeter; and/or (ii) said fibroblast cells and said dermal papilla cells included in said second hydrogel carrier in a ratio of about 8:1 or 6:1 to 2:1 or 1:1 (preferably 5:1 to 3:1) and/or at a combined density (with endothelial cells, if present) of about 5 or 8 million to 15, 20, 25 or 30 million (preferably about 10 million or about 20-25 million) cells per cubic centimeter; and/or (iii) said keratinocytes and said melanocytes included in said third hydrogel carrier in a ratio of about 20:1 or 10:1 to 8:1, 5:1, 3:1 or 2:1 (preferably from 12:1 to 3:1) and/or at a combined density (with immune cells, if present) of about 5 or 8 million to 15, 20, 25, 30 or 35 million (preferably about 10 million or about 20-30 million) cells per cubic centimeter. In some embodiments, endothelial cells are present in the dermis-like layer at a ratio with respect to the fibroblast cells of about 2:1, 1:1 or 1:2. In some embodiments, immune cells are included in an amount of from 1% or 2%, to 10 or 15%, of the total cells in the epidermis-like layer.

Cells may be obtained from established cultures, donors, or a combination thereof. In some embodiments, said live mammalian adipocytes are human adipocytes, said live mammalian fibroblast cells are human fibroblast cells, said live mammalian follicle dermal papilla cells are human follicle dermal papilla cells, said live mammalian keratinocytes are human keratinocytes, said live mammalian endothelial cells are human endothelial cells, said live mammalian immune cells are human immune cells, and/or said live mammalian melanocytes are human melanocytes.

In some embodiments, the construct may further comprise neural cells or precursors thereof in and/or between said first, second and/or third layer (e.g., in a total amount of from 1 or 2 million to 8 or 10 million (preferably 4 to 6 million) cells per cubic centimeter). Neural cells, including precursors thereof, are known. See, e.g., U.S. Pat. Nos. 6,001,654 and 8,785,187.

In some embodiments, the construct has a diameter or width of from 1 to 5 millimeters, or from 3 to 7 millimeters, or from 5 to 10 millimeters, or from 8 to 16 millimeters, or from 10 to 20 millimeters, or from 20 to 50 millimeters, or from 30 to 80 millimeter, or from 50 to 100 millimeters.

Depositing can be carried out by any suitable technique, including, but not limited to, spraying, spreading/painting, coating, etc. In some preferred embodiments, the depositing steps are carried out by printing or bioprinting in accordance with any suitable technique, including both “ink jet” type printing and syringe injection type printing. Apparatus for carrying out such bioprinting is known and described in, for example, Boland et al., U.S. Pat. No. 7,051,654; Yoo et al., US Patent Application Pub. No. US 2009/0208466; and Kang et al., US Patent Application Publication No. US 2012/0089238.

When deposited on an inert substrate, the constructs described above may be removed therefrom and used immediately, or maintained and further propagated on that support in vitro in any suitable culture media. The constructs may be packaged (with or without the support, or transferred to a different support) in a sterile container or package for subsequent use if desired, along with appropriate nutrients and/or culture media.

The support may be porous or non-porous. For example, the support may be a porous filter, membrane or mesh that is permeable to media nutrients for diffusion to the live cells of the construct, e.g., of one or more of the layers.

### 2. Methods of Use in Wound Treatment.

A wound, such as a burn, incision (including surgical incision), abrasion, laceration or the like on a subject may be treated by topically applying a skin construct as described herein to that wound in a treatment-effective amount and/or configuration (e.g., sufficiently covering or overlying the wound to aid in the healing thereof). Depending on the nature of the wound, such as a burn which is not deep, the first “hypodermis-like” layer may not be required. Suitable subjects include both human subjects, and other animal (typically mammalian) subjects (e.g., dogs, cats, cows, pigs, sheep, horses, etc.) for veterinary (including veterinary medicine and pharmaceutical screening) purposes.

In some embodiments, the wound may be a facial wound, such as a wound of the forehead, glabella, nasion, nose (e.g., nasal bridge, rhinion, infatip lobule, supratip, columella, alar-sidewall), nasolabial fold, philtrum, lips, chin, cheek, jaw, ear (e.g., helix, scapha, antihelical fold, antihelix, antitragus, lobule, tragus, concha, fossa), skin surrounding the eye (e.g., eyelid), etc.

In some embodiments, the skin construct may be fabricated on a customized mold made of an inert substrate in order to provide a personalized shape for wound healing. The mold may be fabricated based on clinical image data such as CT data, optionally modified to impart the desired shape and features for the wound healing. As a non-limiting example, the mold may be formed from a polymeric material (e.g., polyurethane), optionally dispensed from a printer as taught herein. In some embodiments, the wound may be the result of a surgery or other medical procedure, such as plastic surgery.

In some embodiments, an epidermis layer is deposited on the inert substrate, a dermis layer is deposited on the epidermis layer, and optionally a hypodermis layer is deposited on the dermis layer (depending on the nature of the wound and the need for the hypodermis in the wound treatment).

In some embodiments, the live skin construct comprising an inert substrate layer is molded to snugly fit onto the complex contour, shape and architecture of facial wounds.

In some embodiments, one or more cell types of the construct are autologous with respect to the subject to be treated. In some embodiments, one or more cell types of the construct are allogenic with respect to the subject to be treated.

### 3. Methods of Use in Compound Testing.

Skin constructs as described herein may be used as an alternative to live animal testing for compound or composition screening (e.g., screening for efficacy, toxicity, penetration, irritation, immune response, or other metabolic or physiological activity). Such testing may be carried out by providing a skin substitute construct as described herein under conditions which maintain constituent cells of that construct alive (e.g., in a culture media with oxygenation); applying a compound or composition to be tested (e.g., a drug candidate, typically provided in a vehicle or carrier, a topical composition such as a soap or cosmetic, etc.) to that construct (e.g., by topical application to said third layer); and then detecting a physiological response (e.g., damage, scar tissue formation, irritation, penetration, cell proliferation, etc.) to said skin substitute construct (e.g., burn, cell death, marker release such as histamine release, cytokine release, changes in gene expression, etc.), the presence of such a physiological response indicating said compound or composition has therapeutic efficacy, toxicity, irritation, penetration, or other metabolic or physiological activity if applied to the skin of a mammalian subject. A control sample of the skin substituted may be maintained under like conditions, to which a control compound or composition (e.g., physiological saline, compound vehicle or carrier) may be applied, so that a comparative result is achieved, or damage can be determined based on comparison to historic data, or comparison to data obtained by application of dilute levels of the test compound or composition, etc.

In some embodiments, the skin construct is formed on and/or provided on an insert configured to be placed into a cell culture dish (e.g., a petri dish, a 2-well plate, a 6-well plate, a 12-well plate, a 24-well plate, 48-well plate, 96-well plate, etc.), such as a cell culture insert. Cell culture inserts are known and described in, e.g., U.S. Pat. Nos. 5,652,142, 5,578,492, 5,468,638, 5,470,473, etc.

The present invention is explained in greater detail in the following non-limiting Examples.

### EXAMPLES

### Example 1: Improved Bioprinted Skin with Seven Cell Types

A bioprinted full-thickness human skin construct was developed having stratified tri-layered structures containing epidermis, dermis and hypodermis. The bioprinted skin construct contained hair follicle appendages, microvasculature, immune cells and pigmentation, and is structurally similar to native human skin.

Full thickness human skin was bioprinted with seven different cell types: keratinocytes, melanocytes, CD14+ monocytes, dermal fibroblasts, dermal microvasculature endothelial cells, follicle dermal papilla cells, and adipocytes. The cells were provided in hydrogel of hyaluronic acid (HA) and gelatin (1% each), and human collagen (10%), cross-linked with a 4-arm poly ethylene glycol cross-linker (HyStem hydrogel kit).

In brief, the hypodermis is printed first; 48 h prior to printing, adipocytes and endothelial cells are co-cultured as spheroids and incorporated into the hydrogel. For dermis printing, 48 h prior to printing, follicle dermal papilla cells and independently, endothelial cells and fibroblasts, are co-cultured as spheroids, then the dermal layer containing fibroblasts, endothelial-fibroblast and follicle spheroids are printed. Finally the epidermal layer containing keratinocytes, melanocytes and CD14+ monocytes is printed on top of the dermis. However, the reverse sequence of layer printing may also be performed.

The printed constructs are cultured under submerged conditions for 4 days and then at the air-liquid interface to facilitate epidermal stratification. In vitro the bioprinted skin constructs showed stratified tri-layer structure with epidermis, dermis and hypodermis.

By 3 weeks, hair follicle organization was observed with outer and inner hair root sheath in vitro. After 56 days in culture, the skin constructs maintained their structural organization and individual cell types were viable and remained localized to their specific region in the construct.

The skin constructs had pigmentation visible to the naked eye (see FIG. 2), showed the presence of immune cells, endothelial cells and had hair follicle structure organization. In particular, at day 56 the epidermis was positive for dendritic cells (DC-SIGN positive) and Langerhans cells (Langerin positive). Hair follicle structure organization included inner and outer root sheaths, as indicated by being cytokeratin 14 positive (outer root sheath) and cytokeratin 71 positive (inner root sheath).

Physiologically, tissues are made up of different kinds of cells that live in close proximity to each other typically in niches. The spheroid culture of cells used in this work may facilitate greater interaction between the different types of cells, contributing to their observed improved maintenance over an extended period of time. The cells are also observed to exhibit greater potential for differentiation to specific structures such as the hair follicles.

In these bioprinted constructs, viability was extended and state of differentiation of the different cells was enhanced over eight weeks. In the case of melanocytes, not only is there continued viability of the cells at eight weeks of culture, but also melanin pigment production is distinctly visible on the bioprinted constructs (FIG. 2). Prior to this modification in culture, pigmentation was observed to significantly decrease after the first week of culture and could not be maintained long-term.

Immune cells are also found to be viable after eight weeks and do not appear to be diminishing in number. Further, they were found to differentiate into dermal dendritic cells in addition to Langerhans cells. In prior work, the immune cells were observed to decrease in number following one week of culture.

The spheroid culture techniques have also shown extended viability of follicle dermal papilla cells and their differentiation into inner and outer root sheath structures in vitro. Additionally, dermal microvasculature endothelial cells are observed to be viable and their presence maintained at the eight week culture period. Though not wishing to be bound by theory, these significantly different effects observed in the bioprinted constructs following incorporation of spheroids of the different cell types maybe due to secreted factors these cells are exposed to in their surrounding microenvironment.

In conclusion, bioprinted skin constructs were bioprinted using human cells and matured in vitro to stratified tri-layered structures containing epidermis with immune cells and pigmentation, dermis with hair follicles, and hypodermis, similar to native human skin.

### Example 2: Further Characterization of Bioprinted Skin Constructs

Staining showed the presence of Langerhans (Langerin+) cells and dermal dendritic cells (DC-SIGN) at Day 5 after bioprinting of constructs with CD14+ monocytes. Inner root sheath (KRT71), outer root sheath (KRT14) and endothelial cells (CD31) were also seen at Day 5.

At Day 21, constructs stained positive for outer root sheath (KRT14) and endothelial cells (CD31). This shows that the bioprinted skin constructs are immune-competent, can form endothelial structures and can support hair-follicle organization. Further, MEL5 staining showed that presence of numerous melanocytes throughout the constructs. Also, there was co-localization of the melanocytes with hair follicle-like structures, suggesting pigmentation in hair follicle-like structures.

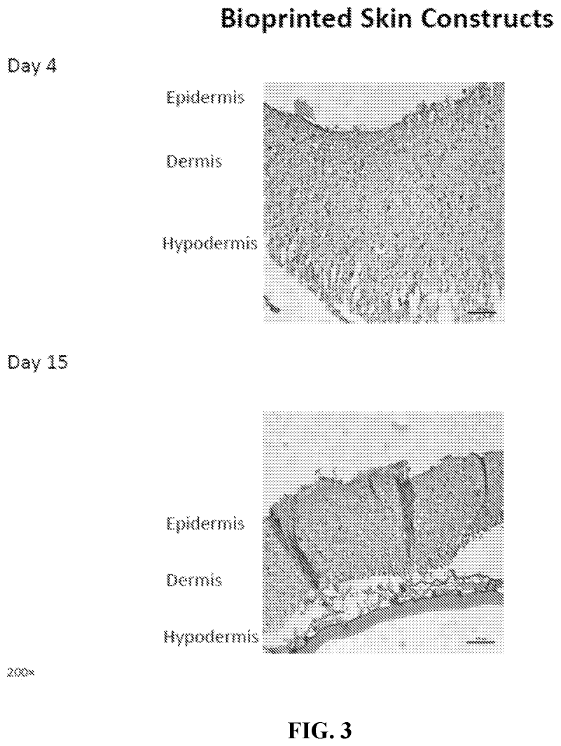

FIG. 3 shows the layered configuration of bioprinted skin constructs at Day 4 and Day 15. Staining indicated the constructs were positive for pan cytokeratin at Day 4 and Day 15.

### Example 3: Bioprinted Skin with Seven Cell Types for Treatment of Burn Wound

Methods: The different cell types are sourced from commercial vendors and expanded in culture to achieve the relevant cell numbers. Each of the cell types that have been expanded in their specific growth media are trypsinized and homogeneously distributed into a cell suspension, which is incorporated into the bioink for layer-by-layer 3D bioprinting. The bioink used for 3D bioprinting consists of hyaluronic acid (3 mg/mL), gelatin (35 mg/mL), glycerol (100 uL/mL), fibrinogen (30 mg/mL), and the cells, which is cross-linked with thrombin (20 uL/mL) post-3D bioprinting. 3D bioprinting of the constructs is done as described previously. For hypodermis printing, adipocytes (30×10⁶ cells/mL) are incorporated into the bioink. For printing of the dermis, fibroblasts (30×10⁶ cells/mL), endothelial cells (15×10⁶ cells/mL) and follicle dermal papilla cells (15×10⁶ cells/mL) as hair follicle spheroids that were prior grown in hanging drop cultures for 48 h, are incorporated into the bioink. For epidermis printing keratinocytes (40×10⁶ cells/mL), melanocytes (8×10⁶ cells/mL), and immune cells (2×10⁶ cells/mL) will be used. The constructs are printed in 3 cm×3 cm dimensions. Post-printing the constructs are matured under submerged culture for four days, after which they are transplanted onto 2.5 cm×2.5 cm excisional wounds on the backs of nude mice. These mice are then followed up to 8 weeks, with time points at each week except week 7.

Preliminary data indicates that the bioprinted skin constructs enhances more rapid wound closure as compared to the bioprinted gel only and wound but no treatment controls. The rate of healing was significantly faster in the bioprinted group compared to the controls. The bioprinted skin facilitated formation of a thicker and stratified epidermis over the wounds compared to the controls.

The foregoing is illustrative of the present invention, and is not to be construed as limiting thereof. The invention is defined by the following claims, with equivalents of the claims to be included therein.

1. An artificial mammalian skin construct, comprising:
optionally, a first (“hypodermis-like”) layer comprising live mammalian adipocytes (e.g., induced pre-adipocytes) and optionally live mammalian endothelial cells (e.g., dermal microvasculature endothelial cells) in a first hydrogel carrier;
a second (“dermis-like”) layer on or directly contacting said first layer, when present, said second layer comprising live mammalian fibroblast cells, live mammalian follicle dermal papilla cells, and optionally live mammalian endothelial cells (e.g., dermal microvasculature endothelial cells) in combination in a second hydrogel carrier; and
a third (“epidermis-like”) layer on or directly contacting said second layer, said third layer comprising live mammalian keratinocytes, live mammalian melanocytes, and live mammalian immune cells (e.g., CD14+ monocytes, Langerhans cells, dermal dendritic cells, or a combination of two of more thereof) in combination in a third hydrogel carrier,
wherein said construct has visible pigmentation (e.g., after 3, 4, 5, 6, 7, or 8 weeks in culture).

2. The construct of claim 1, wherein the live mammalian immune cells of the third layer comprise Langerhans cells and dermal dendritic cells.

3. The construct of claim 1, wherein the hypodermis-like layer, the dermis-like layer, or both, comprise the live mammalian endothelial cells.

4. The construct of claim 1, wherein both the hypodermis-like layer and the dermis-like layer comprise the live mammalian endothelial cells.

5. The construct of claim 1, wherein said construct is a stratified, tri-layered construct.

6. The construct of claim 1, wherein said construct has hair follicle structure organization (inner and outer root sheaths, which may be indicated by being cytokeratin 14 positive and cytokeratin 71 positive) in vitro, and/or are positive for PROMININ-1.

7. The construct of claim 1, wherein said first, second, and/or third hydrogel carriers comprise cross-linked hyaluronic acid (e.g., cross-linked with a polyethylene glycol crosslinker), and/or wherein said first, second and/or third hydrogel carriers optionally further comprise collagen (e.g., at 5, 8, 10, or 15% by weight), and/or gelatin (e.g., at 0.5 1, 2, 3 or 5% by weight).

8. The construct of claim 1, wherein:
(i) said first layer when present has a thickness of from 100, 200 or 300 micrometers up to 400, 600 or 800 micrometers;
(ii) said second layer has a thickness of from 100, 200 or 300 micrometers up to 400, 600 or 800 micrometers;
(iii) said third layer has a thickness of from 100, 200 or 300 micrometers up to 400, 600 or 800 micrometers; and/or
(iv) said construct has a total thickness of from about 200, 400 or 600 micrometers up to 800, 1200 or 1600 micrometers when said first layer is absent, or a total thickness of 300, 600 or 900 micrometers up to 1200, 1800 or 2400 micrometers when said first layer is present.

9. The construct of claim 1, wherein each of said first layer when present, said second layer, and said third layer have overlying surface areas of from 0.5, 1 or 10 square centimeters up to 50, 200 or 400 square centimeters.

10. The construct of claim 1, wherein:
(i) said adipocytes and said endothelial cells, when present, are included in said first hydrogel carrier in a ratio of about 2:1, 1:1, or 1:2 and/or at a combined density of about 1 or 2 million to 8, 10, or 20 million cells per cubic centimeter; and/or
(ii) said fibroblast cells and said dermal papilla cells are included in said second hydrogel carrier in a ratio of about 8:1 or 6:1 to 2:1 or 1:1, with said endothelial cells present in the second hydrogel carrier at a ratio with respect to the fibroblast cells of about 2:1, 1:1 or 1:2, and/or the cells are at a combined density of about 5 or 8 million to 15, 20, 25 or 30 million cells per cubic centimeter; and/or
(iii) said keratinocytes and said melanocytes are included in said third hydrogel carrier in a ratio of about 20:1 or 10:1 to 8:1, 5:1, 3:1 or 2:1 and/or at a combined density of about 5 or 8 million to 15 or 20, 25, 30 or 35 million cells per cubic centimeter, and said immune cells are included in an amount of from 1% or 2%, to 10 or 15%, of the total cells in the epidermis-like layer.

11. The construct of claim 1, wherein: said live mammalian adipocytes are human adipocytes, said live mammalian fibroblast cells are human fibroblast cells, said live mammalian follicle dermal papilla cells are human follicle dermal papilla cells, said live mammalian keratinocytes are human keratinocytes, said live mammalian melanocytes are human melanocytes, said live mammalian endothelial cells are human endothelial cells, and said live mammalian immune cells are human immune cells.

12. The construct of claim 1, wherein said construct is produced by a process comprising:
(a) optionally co-culturing the adipocytes and endothelial cells as spheroids; incorporating the spheroids into the first hydrogel carrier to form a hypodermal bioink; and depositing (e.g., by bioprinting) the hypodermal bioink on a substrate to form the first (hypodermis-like) layer;
(b) culturing the follicle dermal papilla cells as spheroids; independently, co-culturing the endothelial cells and the fibroblasts as spheroids; incorporating the spheroids into the second hydrogel carrier to form a dermal bioink; and depositing (e.g., by bioprinting) the dermal bioink onto the hypodermis-like layer, when present, or onto a substrate when not present, to form the second (dermis-like) layer; and
(c) incorporating the keratinocytes, the melanocytes and the immune cells into the third hydrogel carrier to form an epidermal bioink; and depositing (e.g., by bioprinting) the epidermal bioink onto the dermis-like layer to form the third (epidermis-like) layer,
to thereby form the skin construct.

13. The construct of claim 1, wherein said construct is produced by a process comprising:
(a) incorporating the keratinocytes, the melanocytes and the immune cells into a third hydrogel carrier to form an epidermal bioink; and depositing (e.g., by bioprinting) the epidermal bioink on a substrate to form the third (epidermis-like) layer;
(b) culturing the follicle dermal papilla cells as spheroids; independently, co-culturing the endothelial cells and the fibroblasts as spheroids; incorporating the spheroids into the second hydrogel carrier to form a dermal bioink; and depositing (e.g., by bioprinting) the dermal bioink onto the epidermis-like layer to form the second (dermis-like) layer; and
(c) optionally, co-culturing the adipocytes and the endothelial cells as spheroids; incorporating the spheroids into the first hydrogel carrier to form a hypodermal bioink; and depositing (e.g., by bioprinting) the hypodermal bioink onto the dermis-like layer to form the first (hypodermis-like) layer,
to thereby form the skin construct.

14. The construct of claim 12, wherein the depositing is carried out by bioprinting (e.g., “ink jet” type printing and/or syringe injection type printing).

15. A method of treating a wound on a subject in need thereof, comprising topically applying the skin construct of claim 1 to said wound in a treatment-effective amount and/or configuration, optionally wherein the cells of the construct are autologous or allogeneic.

16. The method of claim 15, wherein said skin construct further comprises an inert mold layer on or contacting said third layer.

17. The method of claim 16, wherein said inert mold layer is dimensioned for custom fit onto a facial wound (e.g., based on scan data).

18. The method of claim 17, wherein said facial wound is a wound of the forehead, glabella, nasion, nose (e.g., nasal bridge, rhinion, infatip lobule, supratip, columella, alar-sidewall), nasolabial fold, philtrum, lips, chin, cheek, jaw, ear (e.g., helix, scapha, antihelical fold, antihelix, antitragus, lobule, tragus, concha, fossa), and/or skin surrounding the eye (e.g., eyelid).

19. The method of claim 15, wherein said inert mold layer comprises polyurethane.

20. The method of claim 15, wherein said inert mold layer is porous.

21. A method of screening a compound or composition for activity when applied to the skin of a mammalian subject, comprising:
providing the skin construct of claim 1 under conditions which maintain constituent cells of said construct alive;
contacting said compound or composition to said construct; and then
detecting a response of said skin construct, the presence of such response indicating said compound or composition is potentially active if applied to the skin of a mammalian subject.

22. The method of claim 21, wherein said response comprises an immune response (e.g., cytokine release).

23. A method of making a skin construct, comprising the steps of:
(a) optionally co-culturing adipocytes and endothelial cells as spheroids; incorporating the spheroids into a first hydrogel carrier to form a hypodermal bioink; and depositing (e.g., by bioprinting) the hypodermal bioink on a substrate to form a first (hypodermis-like) layer;
(b) culturing follicle dermal papilla cells as spheroids; independently, co-culturing endothelial cells and fibroblasts as spheroids; incorporating the spheroids into a second hydrogel carrier to form a dermal bioink; and depositing (e.g., by bioprinting) the dermal bioink onto the hypodermis-like layer, when present, or a substrate when not present, to form a second (dermis-like) layer; and
(c) incorporating the keratinocytes, melanocytes and immune cells into a third hydrogel carrier to form an epidermal bioink; and depositing (e.g., by bioprinting) the epidermal bioink onto the dermis-like layer to form a third (epidermis-like) layer,
to thereby make the skin construct.

24. A method of making a skin construct, comprising the steps of:
(a) incorporating the keratinocytes, melanocytes and immune cells into a third hydrogel carrier to form an epidermal bioink; and depositing (e.g., by bioprinting) the epidermal bioink on a substrate to form a third (epidermis-like) layer;
(b) culturing follicle dermal papilla cells as spheroids; independently, co-culturing endothelial cells and fibroblasts as spheroids; incorporating the spheroids into a second hydrogel carrier to form a dermal bioink; and depositing (e.g., by bioprinting) the dermal bioink onto the epidermis-like layer to form a second (dermis-like) layer; and
(c) optionally, co-culturing adipocytes and endothelial cells as spheroids; incorporating the spheroids into a first hydrogel carrier to form a hypodermal bioink; and depositing (e.g., by bioprinting) the hypodermal bioink onto the dermis-like layer to form a first (hypodermis-like) layer,
to thereby make the skin construct.

25. The method of claim 23, wherein the depositing is carried out by bioprinting (e.g., “ink jet” type printing and/or syringe injection type printing).

26. The method of claim 23, wherein said substrate is an inert substrate.

27. The method of claim 23, wherein said substrate is a wound on a subject (e.g., a human subject) in need of treatment, and optionally wherein the cells are autologous or allogenic.

28. The method of claim 23, wherein the method further comprises culturing the skin construct in vitro under submerged conditions; then culturing at an air-liquid interface, with the epidermal-like layer exposed to air, for a time sufficient to facilitate epidermal stratification of the skin construct.
